(19) United States
(12) Patent Application Publication
(10) Pub. No.: US 2012/0130416 A1
(43) Pub. Date: May 24, 2012

(54) LOW RESISTANCE DILATOR

(75) Inventor: Christopher Joseph Okos, Huntington Beach, CA (US)

(73) Assignee: EDWARDS LIFESCIENCES CORPORATION, Irvine, CA (US)

(21) Appl. No.: 13/320,878

(22) PCT Filed: May 20, 2010
(86) PCT No.: PCT/US10/35572

§ 371 Date: Feb. 9, 2012

Related U.S. Application Data
(60) Provisional application No. 61/180,781, filed on May 22, 2009.

(51) Int. Cl. A61M 29/00

(52) U.S. Cl. 606/194

(57) ABSTRACT

A dilator for use in accessing a vessel or other hollow organ is provided. The dilator has an extruded shaft with a tip portion at the distal end. The tip portion is formed by inserting the distal end portion of an extruded shaft into a die. Typically, the tip portion is molded to be tapered, and has a matte finish on at least a portion of its surface. The non-tapered shaft portion of the dilator may also have a matte finish that is typically formed during the extrusion process. In one embodiment, the matte surface is imparted to the surface of the tip portion after the shaft of the dilator has been extruded, by molding the tip portion in a die that has a matte or unpolished surface. The surface of the molded tip portion is then the inverse of the pattern on the surface of the die.

### BACKGROUND OF THE INVENTION

The Seldinger technique is a medical procedure to obtain safe access to blood vessels and other hollow organs. FIG. 1 generally illustrates the steps of one version of the Seldinger technique. A blood vessel is punctured with a needle assembly (FIG. 1A). Blood is aspirated and the syringe that is attached to the needle is removed (FIG. 1B & C). A guidewire is then advanced through the hollow needle (Fig. E). With the guidewire in place within the vessel, the needle is removed (FIG. 1F). Optionally, a small nick is made with a scalpel immediately adjacent to the guidewire (FIG. 1G). A dilator is advanced over the guidewire to expand the opening into the vessel (FIG. 1H), and is then removed (FIG. 1I). A catheter is advanced over the guidewire and into the vessel (FIG. 1J), and the guidewire is removed to complete the procedure (FIG. 1K).

Considering the dilator of FIGS. 1H and 1I, which is shown in more detail in FIG. 2, a typical dilator has an elongated, tapered shaft with hardware fitted on the proximal end. The shaft is first formed with an extrusion process. The tapered tip portion, at the distal end of the dilator, is formed in a die after the extrusion step. The dilator may be formed from any of a variety of moldable materials, such as polypropylene, latex or teflon. The narrow, tapered tip segment of the dilator permits easy insertion through the skin and into the vessel, with the progressively widening shaft enlarges the opening as the dilator is advanced.

To minimize friction between the shaft of the dilator and the skin, the surface of the dilator tip is commonly made as smooth as possible. This is accomplished in various ways, such as by applying a smooth coating on the tip segment of the dilator. Such finishes tend to be costly, and increase the price of the dilators. Also, the coatings must be approved by regulators, a process that can be expensive and add to the complexity of developing the dilator.

Another approach is to highly polish the die in which the dilator tip segment is molded. The surface of the dilator is thereby made smooth, because the surface of the die from which it is molded is smooth. FIG. 3 is a detailed photo of a portion of the smooth surface of the tip of a dilator of this type.

### BRIEF DESCRIPTION OF THE DRAWINGS

FIG. 1 illustrates the steps of a modified version of the Seldinger technique.

FIG. 2 is a perspective view of one embodiment of a dilator.

FIG. 3 is a detailed photograph of a section of the surface of a smooth dilator tip.

FIG. 4a is a detailed photograph of a small section of the surface of a dilator shaft having a matte finish.

FIG. 4b compares the matte surface of a dilator tip according to one embodiment of the present invention, with a tip portion having a smooth surface.

FIG. 4c is a photograph of a tapered segment of a dilator tip according to one embodiment of the present invention.

FIG. 5 is a chart illustrating non-limiting examples of insertion force required for particular embodiments of latex dilators, with the matte-finish dilator requiring less insertion force than any of three versions of smooth-surface dilators.

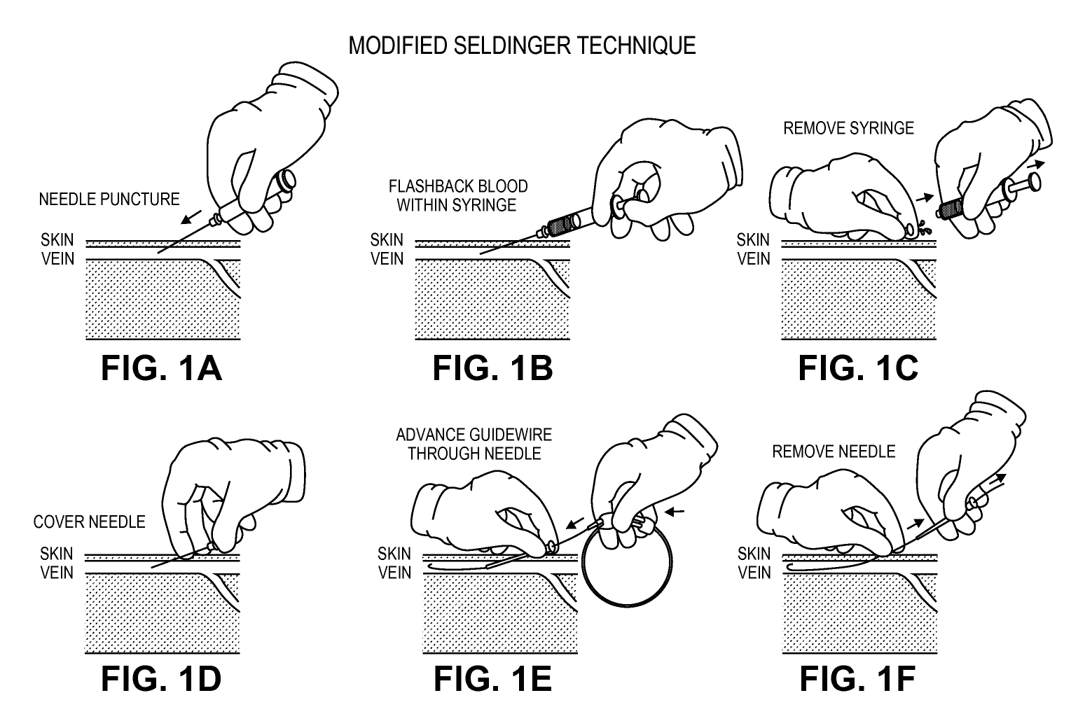

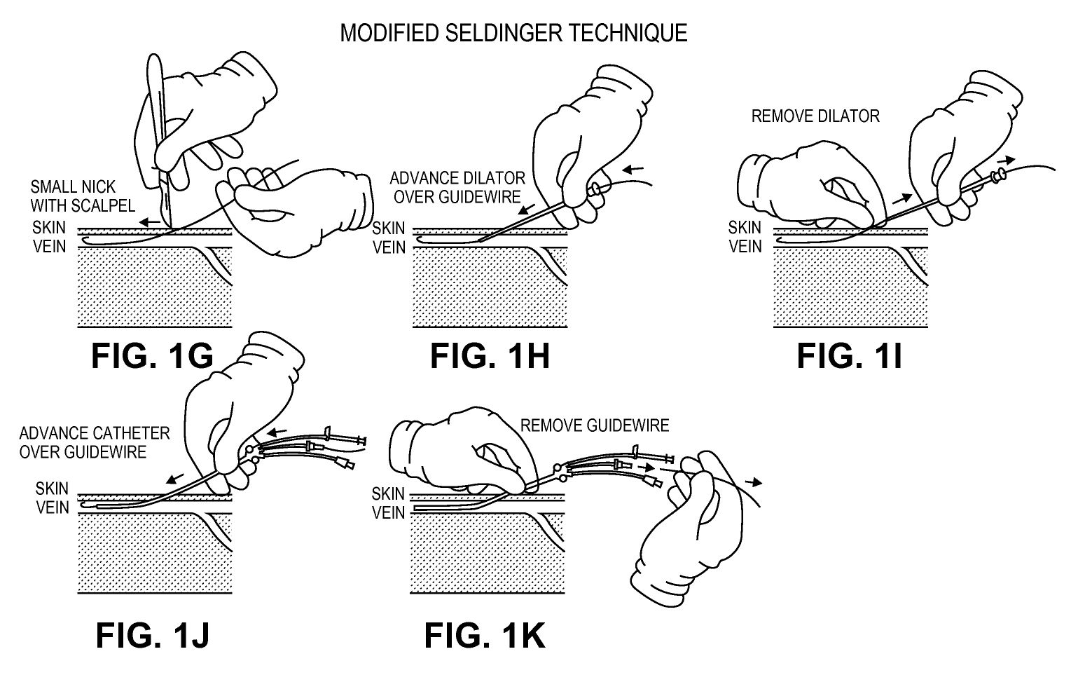

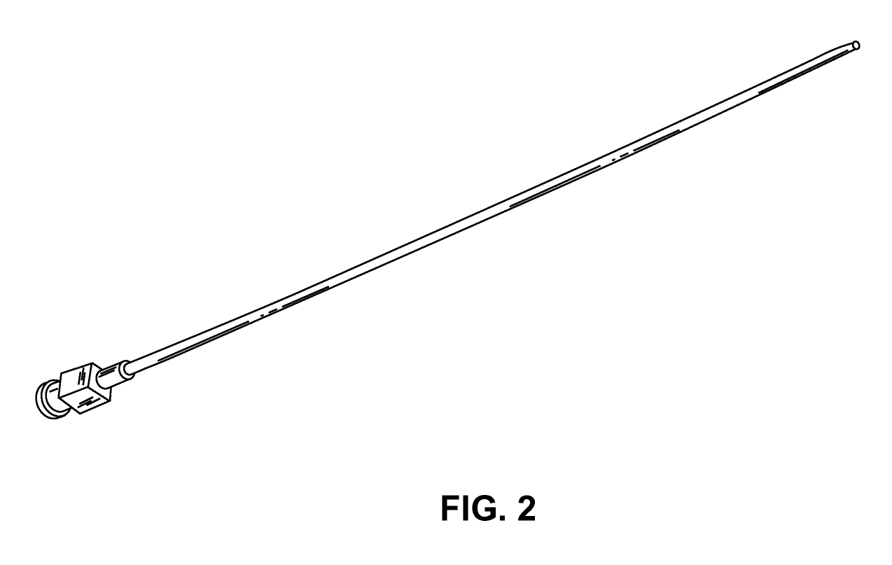

### DETAILED DESCRIPTION OF THE PREFERRED EMBODIMENTS

It has been discovered that a modification to the surface of the tip portion of a dilator shaft can allow the dilator to move with less friction through the skin. In particular, although counter-intuitive, friction between the dilator surface and the skin may be reduced by providing the surface of the dilator tip with a matte finish, rather than with a smooth finish as is common in the art.

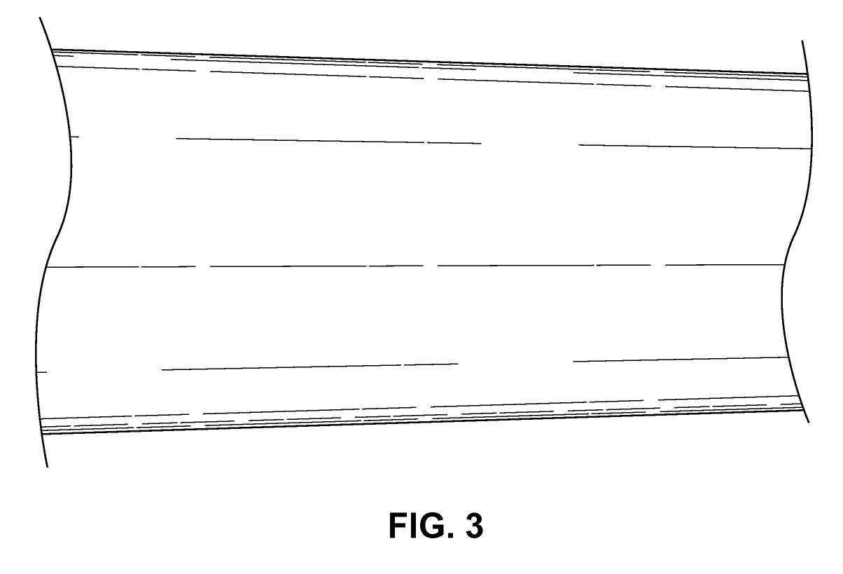

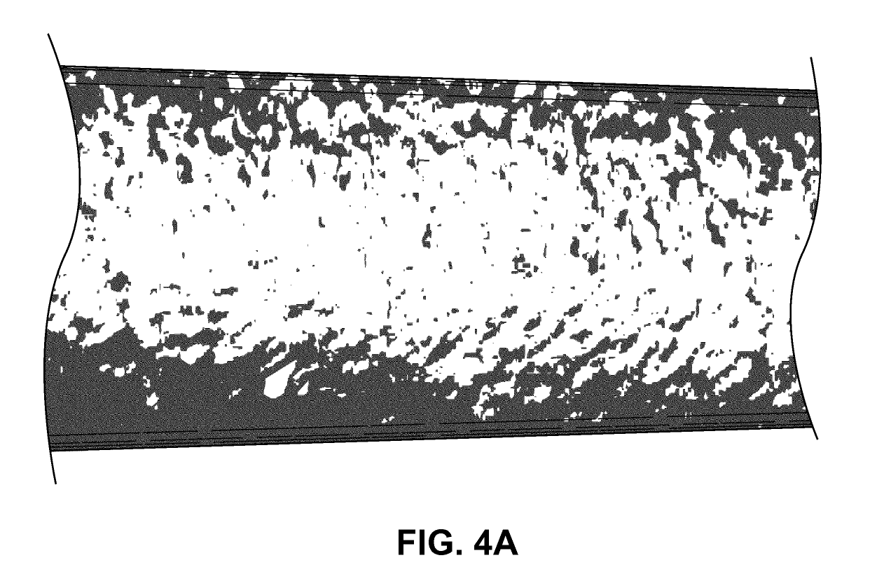

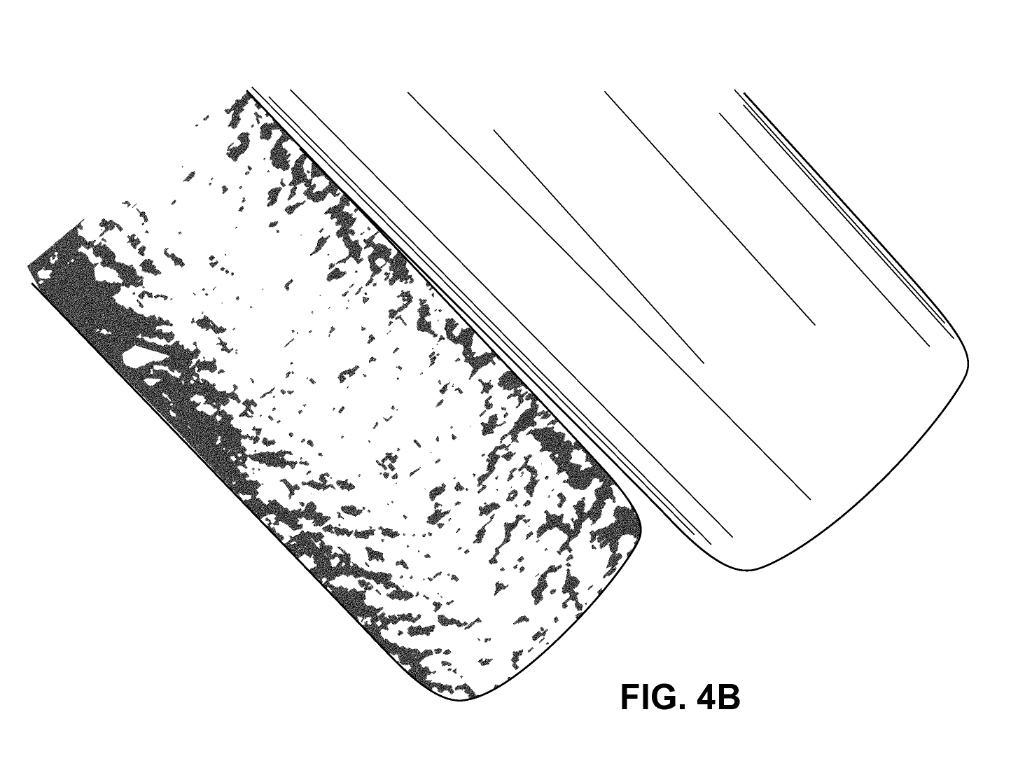

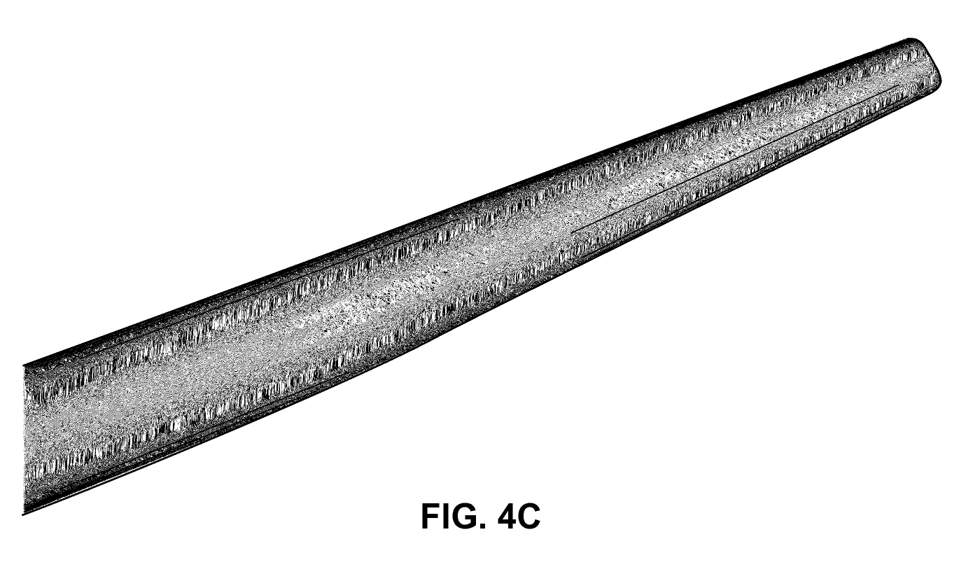

FIG. 3 illustrates a detailed surface photograph of a matte dilator shaft. “Matte” in this context refers to a surface having a roughness of SPI D1, D2 or D3. Alternatively, the surface roughness may be expressed in terms of the scale adopted by the German association of engineers “Vereines Deutscher Ingenieure” (“VDI”). An exemplary table of VDI values is as follows:

VDI3400 Ra = AA = CLA ISO1302 Rt

| VDI 0-45 | μm | μinch | N3-N10 | μm |
|---|---|---|---|---|
| 0 | 0.1 | 4 | N3 | |
| 1 | 0.11 | 4.4 | | |
| 2 | 0.12 | 4.8 | | |
| 3 | 0.14 | 5.6 | | |
| 4 | 0.16 | 6.4 | | |
| 5 | 0.18 | 7.2 | N4 | |
| 6 | 0.2 | 8 | | |
| 7 | 0.22 | 8.8 | | |
| 8 | 0.25 | 10 | | |
| 9 | 0.28 | 11.2 | | |
| 10 | \| 0.32 | 12.8 | | |
| 11 | \| 0.35 | 14 | N5 | |
| 12 | 0.4 | 16 | | 1.6 |
| 13 | 0.45 | 18 | | |
| 14 | 0.5 | 20 | | |
| 15 | 0.56 | 22.4 | | 3.2 |
| 16 | 0.63 | 25.2 | | |
| 17 | 0.7 | 28 | | |
| 18 | 0.8 | 32 | N6 | 5 |
| 19 | 0.9 | 36 | | |
| 20 | 1 | 40 | | |
| 21 | 1.12 | 44.8 | | 7.5 |
| 22 | 1.26 | 50.4 | | |
| 23 | 1.4 | 56 | | |
| 24 | 1.62 | 63 | | 12 |
| 25 | 1.8 | 72 | N7 | |
| 26 | 2 | 80 | | |
| 27 | 2.2 | 88 | | 16 |
| 28 | 2.5 | 100 | | |
| 29 | 2.8 | 112 | | |
| 30 | 3.2 | 125 | N8 | 20 |
| 31 | 3.5 | 140 | | |
| 32 | 4 | 160 | | |
| 33 | 4.5 | 180 | | 25 |
| 34 | 5 | 200 | | |
| 35 | 5.6 | 224 | | |
| 36 | 6.3 | 250 | N9 | 37 |
| 37 | 7 | 280 | | |
| 38 | 8 | 320 | | |
| 39 | 9 | 360 | | 46 |
| 40 | 10 | 400 | | |
| 41 | 11.2 | 448 | | |
| 42 | 12.6 | 500 | N10 | 60 |
| 43 | 14 | 560 | | |
| 44 | 16 | 640 | | |
| 45 | 18 | 760 | | 85 |

Generally speaking, a dilator tip surface according to the present invention has a VDI value of about VDI 12-28. In one preferred embodiment, the surface has a roughness value of VDI 24.

In one non-limiting example of a dilator, strictly for the purposes of illustration, the dilator is 4½ inches long, with the tip portion being about 0.5″ to 0.65″ long. For special applications, the tip may be longer (e.g. 1.0″) or shorter.

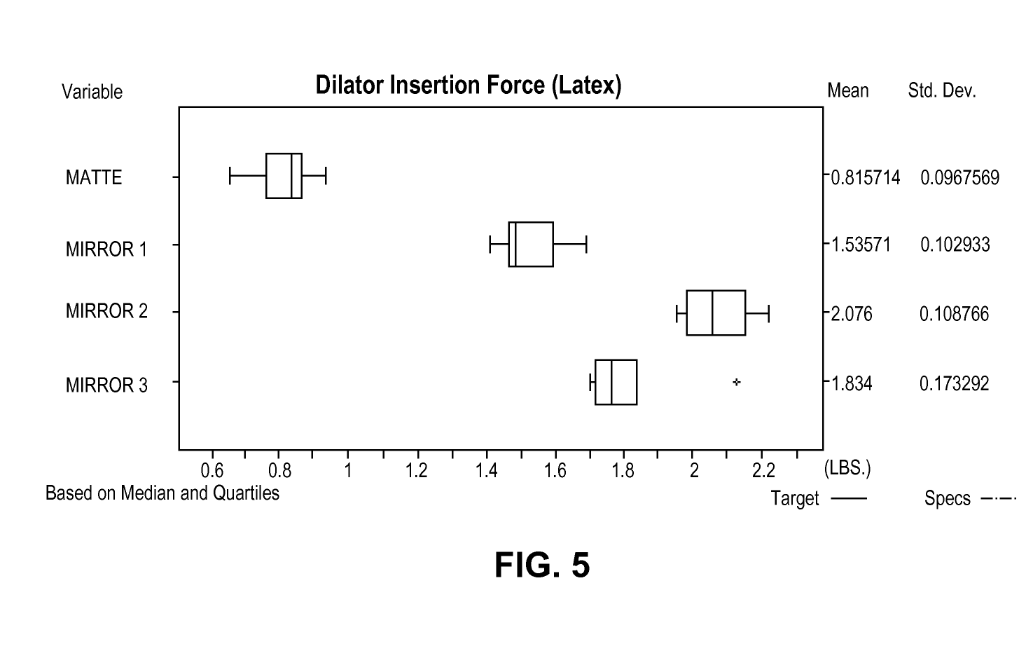

FIG. 5 is a chart illustrating exemplary, non-limiting examples of the insertion force required to insert dilators with different types of surface roughness on the tip segment of the dilator. One particular dilator with a matte finish is found to require an insertion force of less than approximately 0.8 pounds. The chart of FIG. 5 compares this to three different dilators that have a smooth finish on the tip portion. One dilator having a “mirror” finish on the tip was found to require an insertion force of approximately 1.5 pounds. Another was found to require an insertion force of approximately 2.1 pounds, while a third required a force of approximately 1.75 pounds.

As seen in FIG. 5, the dilator with the matte finish on the tip encounters dramatically less friction during insertion than any of the three dilators that have a smooth surface. That is, the dilator with the matte finish tip grips the skin to a lesser extent than a smooth-surfaced dilator tip. This counter-intuitive result is explained, perhaps, in that the rougher finish of the matte surface tip has small indentations, leaving less surface area that comes into contact with the skin than a smooth finish.

A dilator according to the present invention may be formed by inserting the tip portion of an extruded tube into a die. The inner die surface has a matte finish, thereby producing a matte finish on the surface of the molded dilator tip. One embodiment of a die according to the present invention has a fine unpolished EDM (Electrical Discharge Machining) finish. Or, more generally, the molding surface of the die may be of sufficient roughness to produce a surface of a molded polymer dilator tip having a VDI value of between about VDI 12-28.

In one embodiment of the invention, the surface of the die is formed so as to produce a tapered dilator shaft having a matte finish over the entire surface of the tip portion of the dilator. In alternative embodiments, the dilator may be formed so as to have a matte finish on only selected areas of the tip surface, with another type of surface (such as a polished surface or, alternatively, a surface even rougher than matte) at other desired locations.

The shaft portion of the dilator may also have a matte finish. This can be accomplished during the extrusion process by adjusting extrusion variables, such as temperature, speed and/or other variables of the extruding process. In one embodiment, the shaft portion of the dilator is given a “frosted,” non-smooth finish.

While the foregoing discusses a preferred embodiment in which the shaft of the dilator is molded, in an alternative the dilator may be made of a metal, for example, having a matte tip surface formed on the metal through an EDM or other process known in the art.

While particular forms of the invention have been illustrated and described, it will be apparent that various modifications can be made without departing from the spirit and scope of the invention. For example, in some embodiments the matte finish may alternatively be provided with a matte coating, rather than or in addition to the die and/or extrusion processes discussed above. Accordingly, it is not intended that the invention be limited to the specific illustrative embodiments discussed herein.

What is claimed is:

1. A dilator for use in accessing a blood vessel, the dilator having a shaft portion and a tip portion, the shaft and tip portions each having a respective outer surface, wherein at least some of the outer surface of the tip portion has a matte finish.

2. (canceled)

3. A dilator as defined in claim 2, wherein the matte finish is molded onto an outer surface of the tip portion of the dilator.

4. A dilator as defined in claim 2, wherein the tapered tip portion has an outer surface and in which the full outer surface of the tip portion has a matte finish.

5. A method of obtaining safe access to a vessel, comprising the step of dilating an opening with a dilator having a tip portion and a matte finish on the tip portion.

6. A method of manufacturing a dilator comprising the steps of:
having a die with an unpolished surface;
inserting an end portion of an extruded tube into the die; and
molding the end portion of the tube into a tapered tip portion having a matte finish.

7. A method of manufacturing a dilator as defined in claim 6, wherein the method also comprises the step of extruding the tube in an that imparts a matte or other non-smooth finish to the full outer surface of the tube.

8-10. (canceled)
